(19) United States
(12) Patent Application Publication
(10) Pub. No.: US 2022/0249401 A1
(43) Pub. Date: Aug. 11, 2022

(54) DIETARY SUPPLEMENT FORMULATED BASED ON ALL-TRANS FORM OF MENAQUINONE-7

(71) Applicant: NUTRIOMICS LIMITED, Te Awamutu (NZ)

(72) Inventor: Aydin Berenjian, State College, PA (US)

(21) Appl. No.: 17/412,851

(22) Filed: Aug. 26, 2021

(30) Foreign Application Priority Data
Feb. 10, 2021 (NZ) 772850
Feb. 10, 2021 (NZ) 772851

(51) Int. Cl. A61K 31/122; A61K 33/30; A61K 33/34; A61K 33/32; A61K 33/06; A61K 33/26; A61K 33/24; A61K 31/19; A61K 31/375; A61K 31/4188; A61K 31/593; A61K 33/42; A61K 33/04

(52) U.S. Cl. CPC A61K 31/122; A61K 33/30; A61K 33/34; A61K 33/32; A61K 33/06; A61K 33/26; A61K 33/04; A61K 31/19; A61K 31/375; A61K 31/4188; A61K 31/593; A61K 33/42; A61K 33/24

(57) ABSTRACT

The present invention is directed to a dietary supplement comprising a trans form of Menaquinone-7 (MK-7). In some embodiments, the dietary supplement may further comprise at least one chondroprotective agent. In some embodiments, the dietary supplement comprises metal ions selected from the group consisting of zinc ions, copper ions, manganese ions, magnesium ions, iron ions, chromium ions, calcium ions, and combinations thereof.

### CROSS-REFERENCES TO RELATED APPLICATIONS

This application claims benefit of New Zealand patent number 772,850, filed Feb. 10, 2021, and of New Zealand patent number 772,851, filed Feb. 10, 2021, the specifications of which are incorporated herein in their entirety by reference.

### TECHNICAL FIELD

The present invention is in the field of dietary supplements with a synergistic action to preserve and help with growth, and repair of bone and joint connective tissue.

### BACKGROUND OF THE INVENTION

Joint stiffness and pain can arise from intense physical activity, such as the cumulative effects of long-term mechanical stresses experienced during exercise. In addition, joint pain may come from traumatic injuries, such as sprains, fractures and dislocation caused. Joint pain may also arise from arthritic diseases, such as rheumatoid arthritis and bone long-term effects caused. In addition to joint pain, joint disorders may also many abnormalities and loss of movement.

Treatment of joint pain depends on its cause and severity. For repeated mechanical stress due to more moderate joint pain, the easiest way is to relieve stress is by regulating the exercise intensity or duration. For severe joint pain, nonprescription non-steroidal anti-inflammatory drugs (NSAIDs), such as ibuprofen and naproxen sodium are useful. In more severe cases, steroids such as prednisone or cortisone can be used. However, they pose some side effects such as high blood pressure, weight gain and swelling of the face. Medication like etanercept or Adulimo mAb (adulimumab) are the last line medication strategy for chronic arthritic diseases (rheumatoid arthritis) while causing serious side effects like respiratory tract infection, rash and headache. Finally, there is surgical intervention such as cartilage transplant or joint replacement surgery to relieve symptoms. All of the above treatments can solve the pain and limit further damage to the joints, but are almost useless for the promotion of joint tissue repair.

An alternative approach to help the joint pain is through the use of nutritional supplements that stimulate the growth, repair and maintenance of bone and joint connective tissue. One class of supplements includes components of joint connective tissue such as collagen, glucosamine, hyaluronic acid, avocado/soybean unsaponifiables, and chondroitin. Other supplements act as catalysts or supply raw materials for bone and connective tissue synthesis these are mainly S-adenosylmethionione (SAM), methylsulfonylmethane (MSM), and other vitamins and minerals such as Vitamin C, manganese, magnesium, zinc, calcium, iron, and Vitamin B12.

While some of these supplements provide some relief, they typically only address a limited subset of nutritional issues that impact overall joint health. A need therefore exists for a nutritional supplement that can be utilized to improve overall joint health. Compositions containing glucosamine are known to be beneficial to both humans and animals that suffer from osteoarthritis pain. Since glucosamine is a precursor for glycosaminoglycans, and glycosaminoglycans are a major component of joint cartilage, supplemental glucosamine may help to rebuild cartilage and treat arthritis. Commonly sold forms of glucosamine are glucosamine sulfate and glucosamine hydrochloride. Glucosamine is often sold in combination with other supplements such as chondroitin sulfate and methylsulfonylmethane. Generally, vitamin C is needed together with glucosamine sulfate.

Chondroitin sulfate is a sulfated glycosaminoglycan (GAG) composed of a chain of alternating sugars (N-acetyl-galactosamine and glucuronic acid). It is usually found attached to proteins as part of a proteoglycan. Chondroitin sulfate is a major structural component of cartilage and provides much of its resistance to compression. Most of these supplements derive the Chondroitin Sulfate from bovine cartilage or velvet deer antler or shark cartilage.

Avocado/soybean unsaponifiables (ASU) are natural vegetable extracts made from avocado and soybean oils, consisting of the leftover fraction (approximately 1%) that cannot be made into soap after saponification. ASU is composed of one third avocado and two thirds soybean unsaponifiables. The major components of ASU are phytosterols β-sitosterol, campesterol, and stigmasterol, which are rapidly incorporated into cells. The sterol contents of ASU preparations are the primary contributors to biological activity in articular chondrocytes. Preclinical in vitro and in vivo studies have demonstrated that ASUs have beneficial effects on promoting cartilage repair.

Collagen is an essential and major component of muscles, tendons, cartilage, ligaments, joints and blood vessels in humans or animals. There are three main types of collagen: I, II and III. Types I and III are primarily found in skin, tendon and bone. Collagen is an unusual protein, in that the, proportion of glycine residues is nearly one-third, which is unusually high in comparison to other typical proteins. Praline is also present to a much greater extent in collagen than in most other proteins. Moreover, collagen contains two amino acids, 4-hydroxyproline and 5-hydroxylysine, that are found in very few other proteins. The amino acids sequence of collagen is remarkably regular, nearly every third amino acids being glycine. Several patents are related to producing collagen fibers from animal tissues and hydrolyzing the collagen. In contrast, type II is found predominantly in articular cartilage.

Methylsulfonylmethane (MSM, or dimethylsulfone) is an organic sulfur compound belonging to a class of chemicals known as sulfones. It occurs naturally in some primitive plants and is present in small amounts in many foods and beverages. MSM is also known as dimethylsulfone, or DMSO2, a name that reflects its close chemical relationship to dimethyl sulfoxide (DMSO), which differs only in the oxidation state of the sulphur atom. MSM is the primary metabolite of DMSO in humans, and it shares some of the properties of DMSO.

It can be seen that tissue maintenance and building is a complex process that requires anti-inflammatory compounds, collagen generating compositions as well as trace minerals and vitamins at the same time to provide synergistic effect to preserve and rebuild joint tissue. A need, therefore, exists for a nutritional supplement that can be utilized to improve overall joint and bone health.

### DETAILED DESCRIPTION OF THE INVENTION

The term “joint disorders” can be taken to include but not be limited to

a) osteoarthritis,

b) joint effusion,

c) joint erosion,

d) joint inflammation and pain,

e) joint calcification

f) the reduction or inhibition of metabolic activity of chondrocytes,

g) the reduction or inhibition of enzymes that degrade cartilage,

Menaquinone-7 (MK-7), sub-type of Vitamin K2 (Menaquinones), is a nutrient our body needs. MK-7 can promote joint health through different mechanisms including cartilage decalcification, promoting bone health and reducing the inflammation. Articular cartilage calcification and calcium crystal deposition contribute to Arthritis. Matrix Gla Protein (MGP) is responsible to inhibit soft tissue calcification which is MK-7 dependent and in absence of this vitamin have impaired capacity of decalcification. Therefore, MK-7 plays a crucial role in activation of MGP. It also plays a key role in taking calcium from the blood circulation and bind it to the bone matrix that in turn makes the skeleton stronger and less susceptible to fracture. The health of your bones is a big part of the health of your joints as your joints usually connect the ends of two bones together.

Another factor in joint health that is not widely known is chronic inflammation which places stress on your joints at all times and makes it extremely difficult for your joints to recover, leading to extra wear and tear on the joints over time. MK-7 has an anti-inflammatory activity by suppressing nuclear factor κB (NF-κB) signal transduction and to exert a protective effect against oxidative stress by blocking the generation of reactive oxygen species. MK-7 can exist as cis and trans isomers. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”).

The chemical structure of MK-7 influences its ability to interact with subcellular structures, and thus determines its biological activity. Therefore, only the all-trans form of MK-7 is biologically significant. Besides its role as a cofactor for the γ-glutamyl carboxylase enzyme, all-trans form of MK-7 only has an anti-inflammatory activity by suppressing nuclear factor κB (NF-κB) signal transduction and to exert a protective effect against oxidative stress by blocking the generation of reactive oxygen species. Among the various aspects of the invention, the dietary supplement includes the trans form of MK-7 that can exert a protective role in both the inflammatory and mineralization processes as shown in FIG. 1.

To summarize, the trans MK-7 will

1) inhibit cartilage calcification

2) reduce the chronic inflammation and

3) promote bone density by binding calcium to the bone matrix.

It should be noted that some embodiments of the present invention may include the cis form of MK-7, possibly through contamination of the trans MK-7 supply. If so, it should be noted that while the cis form of MK-7 is not active with regard to the present invention, it does not inhibit the efficacy thereof.

Another aspect of the invention encompasses a method for promoting at least one of pain relief, growth, repair, and/or maintenance of bone and/or joint tissue in a mammalian subject. The method comprises administering to the mammalian subject a dietary supplement comprising at least one joint connective tissue and catalysts for bone and connective tissue synthesis. The supplement may be administered as a paste, chewable flavored tablet, capsule, or a powder appointed to be admixed with the food products.

In preferred embodiments, the supplement properly will include additional excipients that will aid to balance metabolic needs for efficacy. Other aspects and feature of the invention will be in part apparent and in part pointed out hereinafter. Preferably, the present invention provides a mammalian supplement formulation that includes vitamins and minerals, as well as formula components that protect and/or build joint and bone tissues. In one embodiment of the present invention, at least one chondroprotective agent including but not limited to eggshell membrane powder, green lipped mussel powder, extract from avocado and soybean or combinations thereof.

For example, joint building ingredient, glucosamine sulphate, needs a substantial quantity of ascorbic acid or vitamin C. However, the vitamin C of the composition is exhausted by the oxidation process of the glucosamine sulfate. More vitamin C is needed for the general upkeep. Calcium is needed for cross-linking cartilage tissue and is provided in the mineral content in biologically usable form. The anti-oxidants provided prevent free radical damage, a key factor in preserving joints. Preferably, the present invention provides dietary supplements having a balanced mixture of key compounds that promote the growth, repair, and maintenance of mammalian bone and joint connective tissue.

In accordance with one aspect of the present invention, the dietary supplements may be administered to a mammalian subject to prevent several joint disorders or indications, including but not be limited to

h) osteoarthritis,

i) joint effusion,

j) joint erosion,

k) joint inflammation and pain,

I) the reduction or inhibition of metabolic activity of chondrocytes,

m) the reduction or inhibition of enzymes that degrade cartilage,

the reduction or inhibition of the production of hyaluronic acid.

The dietary supplements may also be administered to a mammalian subject to decrease degradation of articular cartilage or disorders or indications resulting from degradation of articular cartilage. The dietary supplements of the invention may include a combination of at least trans form of MK-7 and at least one chondroprotective agent. Optionally, the dietary supplement may include at least one additional ingredient selected from the group consisting of vitamins, minerals, amino acids, antioxidants, essential fatty acids, and herbs to help with better MK-7 functionality. Each of these ingredients is described in details hereinafter.

The dietary supplement of the invention may include at least one metal ion. The metal ions may be selected from the group consisting of minerals including, without limitation, calcium, iron, chromium, cooper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In an exemplary embodiment, the mineral may be a form of calcium. Suitable forms of calcium include calcium alpha-ketoglutarate, calcium acetate, calcium alginate, calcium ascorbate, calcium aspartate, calcium caprylate, calcium carbonate, calcium chelates, calcium chloride, calcium citrate, calcium citrate malate, calcium formate, calcium glubionate, calcium glucoheptonate, calcium gluconate, calcium glutarate, calcium glycerophosphate, calcium lactate, calcium lysinate, calcium malate, calcium orotate, calcium oxalate, calcium oxide, calcium pantothenate, calcium phosphate, calcium pyrophosphate, calcium succinate, calcium sulfate, calcium undecylenate, coral calcium, dicalcium citrate, dicalcium malate, dihydroxycalcium malate, dicalcium phosphate, and tricalcium phosphate.

An “effective amount” of a mineral typically quantifies an amount at least about 10% of the New Zealand Ministry of Health Recommended Daily Allowance (“RDA”) of that particular mineral for a subject. It is contemplated, however, that amounts of certain minerals exceeding the RDA may be beneficial for certain subjects. For example, the amount of a given mineral may exceed the applicable RDA by 500% or more.

The dietary supplements of the invention may include at least one chondroprotective agent. Chondroprotective agents suitable for use in the invention generally improve chondrocyte function. Without being bound to any particular theory, suitable chondroprotective agents may improve chondrocyte function by one or more of the following mechanisms: 1) inhibiting cartilage degradation; 2) preventing fibrin formation in the subchondral and synovial vasculature; and 3) stimulating chondrocyte synthesis of collagen and proteoglycans, as well as synoviocyte production of hyaluronan.

In one embodiment, the chondroprotective agent is a natural eggshell membrane which comprises concentrated eggshell membrane powder. The daily dosage of this compound may range from about 100 mg to about 1000 mg and more.

In another embodiment, the chondroprotective agent is a green lipped mussel powder. The daily dosage of such an extract may from about 100 mg to about 3000 mg for a lipid extract or higher for the freeze-dried powder.

Optionally, the dietary supplement of the invention may include one or more vitamins. Suitable vitamins for use in the dietary supplement include vitamin C, vitamin E, vitamin B12, vitamin K2, riboflavin, niacin, vitamin D3, vitamin B6-12, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin.

An “effective amount” of a vitamin typically quantifies an amount at least about 10% of the New Zealand Ministry of Health Recommended Daily Allowance (“RDA”) of that particular vitamin for a subject. It is contemplated, however, that amounts of certain vitamins exceeding the RDA may be beneficial for certain subjects. For example, the amount of a given vitamin may exceed the applicable RDA by 500% or more.

The dietary supplement may optionally include from one to several amino acids. Suitable amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine or their hydroxy analogs.

In certain embodiments, the amino acid will be selected from the essential amino acids. An essential amino acid is generally described as one that cannot be synthesized de novo by the organism, and therefore, must be provided in the diet. By way of non-limiting example, the essential amino acids for humans include: L-histidine, L-isoleucine, L-leucine, L-lysine, L-methionine, L-phenylalanine, L-valine and L-threonine.

The dietary supplement may include one or more suitable antioxidants. Non limiting examples of antioxidants include ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, p-coumaric acid, curcurin, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, propyl gallate, polyphosphates, quercetin, trans-resveratrol, rosmarinic acid, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, zeaxanthin, or combinations thereof.

Natural antioxidants that may be used in the dietary supplement include, but are not limited to, apple peel extract, blueberry extract, carrot juice powder, clove extract, coffeeberry, coffee bean extract, cranberry extract, eucalyptus extract, ginger powder, grape seed extract, green tea, olive leaf, parsley extract, peppermint, pimento extract, pomace, pomegranate extract, rice bran extract, rosehips, rosemary extract, sage extract, tart cherry extract, tomato extract, tumeric, and wheat germ oil.

A variety of commonly used excipients in dietary supplement formulations may be selected on the basis of compatibility with the active ingredients. Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, taste masking agents, an effervescent disintegration agent, and combinations of any of these agents.

In one embodiment, the excipient is a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof. The polypeptide may be any arrangement of amino acids

In another embodiment, the excipient may be a filler. Suitable fillers include carbohydrates, inorganic compounds, and polyvinylpirrolydone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth.

In another embodiment, the excipient may be an effervescent disintegrant. By way of non-limiting example, suitable effervescent disintegrants include sodium bicarbonate in combination with citric acid and sodium bicarbonate in combination with tartaric acid. The excipient may comprise a preservative. Suitable examples of preservatives include antioxidants, such as a-tocopherol or ascorbate, and antimicrobials, such as parabens, chlorobutanol or phenol.

In another embodiment, the excipient may include a diluent. Diluents suitable for use include pharmaceutically acceptable saccharide such as sucrose, dextrose, lactose, microcrystalline cellulose, fructose, xylitol, and sorbitol; polyhydric alcohols; a starch; pre-manufactured direct compression diluents; and mixtures of any of the foregoing. The excipient may include flavors. Flavors incorporated into the outer layer may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, sylitol, and the like.

In another embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

Generally speaking, the dietary supplement may include a mixture of trans form of MK-7 with any composition as described in l(a)(b)(c)(d)(e)(f)(g)(h) or (i). The type and amount of ingredients forming a given dietary supplement may vary greatly depending upon the mammalian subject. The dietary supplement can, in accordance with generally known methods, be formulated to meet the needs of several mammalian subjects. For example, the mammalian subject may be a human or an animal. It is contemplated, if appropriate, that one or more of the ingredients forming the dietary supplement of the present invention can exist in tautomeric, geometric or stereoisomeric forms without departing from the scope of the invention.

The dietary supplements detailed herein may be manufactured in one or several dosage forms. Suitable dosage forms include a tablet, including a suspension tablet, a chewable tablet, an effervescent tablet or caplet; a pill; a powder such as a sterile packaged powder, a dispensable powder, and an effervescent powder; a capsule including both soft or hard gelatin capsules or non-animal derived polymers, such as hydroxypropyl methylcellulose capsules or pullulan; a lozenge; a sachet; a sprinkle; a reconstitutable powder or shake; a troche; pellets; granules; liquids; suspensions; emulsions; or semisolids and gels. Alternatively, the dietary supplement may be incorporated into a food product or powder for mixing with a liquid, or administered orally after only mixing with a non-foodstuff liquid. As will be appreciated by a skilled artisan, the dietary supplements, in addition to being suitable for administration in multiple dosage forms, may also be administered with various dosage regimens.

The amount and types of ingredients and other excipients useful in each of these dosage forms based on the required efficacy. It should be recognized that where a combination of ingredients and/or excipient, including specific amounts of these components, is described with one dosage form that the same combination could be used for any other suitable dosage form.

Example 1—In one exemplary embodiment, a supplement regime in accordance with the present invention could include the following.

| Component | Dosage | |
|---|---|---|
| MK-7 | 80-120 | mg/day |
| Collagen | 500-10.000 | mg/day |
| Glucosamine | 800-2000 | mg/day |
| Hyaluronic acid | 200-1000 | mg/day |
| Chondroitin | 500-1500 | mg/day |
| S-adenosylmethionione | 600-1600 | mg/day |
| Methylsulfonylmethane | 800-3000 | mg/day |
| Glucosamine | 800-2000 | mg/day |

Example 2—The role of the trans form of MK7 in expression of MGP was measured as compared to control samples. Chondrocyte cells were treated with 0.01% (w/w) trans form of vitamin MK7 and results were reported as fold change in gene expression relative to control conditions.

MGP expression was elevated by 21% in the presence of trans form of MK7 as compared to the control samples as can be concluded from FIG. 2. This observation demonstrates the active role of trans form of vitamin MK7 in stimulating the level of mRNA encoding MGP. MGP gene expression found to actively be regulated in the presence of trans form of MK7.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows the chemical structure of the all-trans form of Menaquinone-7.

FIG. 2 shows a graph of MGP expression.

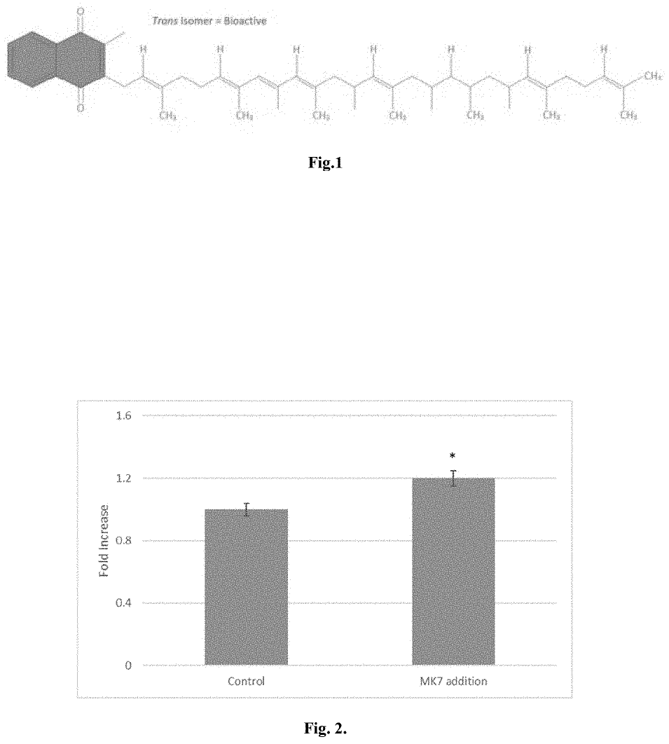

### REFERENCES

Altinel L, Sahin O, Kose K C, Bas O, Ozen O A, Saritas Z K, et al. Healing of osteochondral defects in canine knee with avocado/soybean unsaponifiables: a morphometric comparative analysis. Eklem Hastalik Cerrahisi. 2011; 22(1):48-53.
Andrew M T. arthritis in animals with dietary supplements. U.S. Pat. No. 6,524,609B1.
Boileau C, Martel-Pelletier J, Caron J, Msika P, Guillou G B, Baudouin C, et al. Protective effects of total fraction of avocado/soybean unsaponifiables on the structural changes in experimental dog osteoarthritis: inhibition of nitric oxide synthase and matrix metalloproteinase-13. Arthritis Res Ther. 2009; 11(2):R41.
Christensen R, Bartels E M, Astrup A, Bliddal H. Symptomatic efficacy of avocado-soybean unsaponifiables (ASU) in osteoarthritis (OA) patients: a meta-analysis of randomized controlled trials. Osteoarthritis Cartilage. 2008; 16(4):399-408.
Cindy W. Nutritional supplements. US20070065456A1.
Dinubile N A. A potential role for avocado- and soybeanbased nutritional supplements in the management of osteoarthritis: a review. Phys Sportsmed. 2010; 38(2):71-81.
Gabay O, Gosset M, Levy A, Salvat C, Sanchez C, Pigenet A, et al. Stress-induced signaling pathways in hyalin chondrocytes: inhibition by avocado-soybean unsaponifiables (ASU). Osteoarthritis Cartilage. 2008; 16(3):373-84.
Jeremy D, Moore T R, Hampton R H. Dietary supplements for promotion of growth. repair, and maintenance of bone and joints, U.S. Pat. No. 8,968,791B2,
John M S, Hill C M, Use of eggshell membrane formulations to alleviate joint pain. WO2011062953A1.
Kevin S. Cartilage enhancing food supplements and methods of preparing the same, WO2004004686A2.
Lippiello L, Nardo J V, Harlan R, Chiou T. Metabolic effects of avocado/soy unsaponifiables on articular chondrocytes. Evid Based Complement Alternat Med. 2008; 5(2):191-7.
Marni M H. Joint preserving nutritional vitamin, mineral and herbal pet supplement. U.S. Pat. No. 8,703,174B2.
Nathaniel D, Albert D M, Laberge J, Campisi M, Demaria A, Vasserot C, Kim O, Jeon J, Elisseeff J, Kirkland T, Tchkonia, Yi Z, Treatment of joint pain. U.S. Ser. No. 10/130,628B2.
Patricia M P, Rukmini C, Reddy S, Cherukuri V, Mohammed M, Methods for treating joint inflammation, pain, and loss of mobility. U.S. Pat. No. 6,902,739B2.
Paul T Q, Cees V. Vitamin K2 containing food product. CA2347387C.
Shearer M J, Newman P. Metabolism and cell biology of vitamin K. Thromb Haemostasis. 2008, 100(10):530-547.
Suketsune I, Takehito K. Medicine and functional food. JP2006169205A.
Szterk A, Bus K, Zmysłowski A, Ofiara K. Analysis of Menaquinone-7 Content and Impurities in Oil and Non-Oil Dietary Supplements. Molecules. 2018, 23(5):1056.

What is claimed is:

1. A dietary supplement comprising a trans form of Menaquinone-7 (MK-7).

2. The dietary supplement of claim 1, further comprising at least one chondroprotective agent.

3. The dietary supplement of claim 1, further including at least one ingredient selected from the group consisting of vitamins, minerals, amino acids, antioxidants, and anti-inflammatory agents.

4. The dietary supplement of claim 1, further comprising metal ions selected from the group consisting of zinc ions, copper ions, manganese ions, magnesium ions, iron ions, chromium ions, calcium ions, and combinations thereof.

5. The dietary supplement of claim 4, wherein the metal ions are selected from the group consisting of manganese ions, zinc ions, and copper ions, wherein the dietary supplement further comprises the ligand 2-hydroxy-4-methylthiobutanoic acid.

6. The dietary supplement of claim 1, further comprising vitamin C, biotin, vitamin D3, selenium, calcium, phosphate, and 2-hydroxy-4-methylthiobutanoic acid.

7. The dietary supplement of claim 1, further comprising dried molasses, flax oil, yeast culture, and alfalfa meal.

8. The dietary supplement of claim 1, further comprising magnesium, vitamin C, selenium, calcium, and vitamin D3

9. A medicament for the therapeutic or prophylactic treatment of joint conditions, comprising the trans-form of Menaquinone-7 (MK-7).

10. A method for promoting at least one of pain relief, growth, repair, or maintenance of bone or joint tissue in a mammalian subject, the method comprising administering to the mammalian subject the medicament of claim 9.

11. The method of claim 10, wherein the medicament comprises the all-trans form of MK-7 and at least one chondroprotective agent.

12. The method of claim 10, wherein the medicament further comprises at least one ingredient selected from the group consisting of vitamins, minerals, metal ions, amino acids, antioxidants, and anti-inflammatory agents.

13. The method of claim 12, wherein the metal ions are selected from the group consisting of zinc ions, copper ions, manganese ions, magnesium ions, iron ions, chromium ions, calcium ions, and combinations thereof.

14. The method of claim 12, wherein the metal ions are selected from the group consisting of manganese ions, zinc ions, and copper ions, and the medicament additionally comprises the ligand 2-hydroxy-4-methylthiobutanoic acid

15. The method of claim 10, wherein the mammalian subject is selected from the group consisting of animals and humans.

16. The method of claim 10, wherein the mammalian subject has a joint-related indication.

17. The method of claim 16, wherein the joint-related indication is selected from the group consisting of osteoarthritis, rheumatoid arthritis, psoriatic arthritis, joint effusion, joint inflammation or pain, synovitis, lameness, post-operative arthroscopic surgery, deterioration of proper joint function, the inhibition of metabolic activity of chondrocytes, and the inhibition of the production of hyaluronic acid.
